(19) United States
(12) Patent Application Publication
(10) Pub. No.: US 2004/0219608 A1
(43) Pub. Date: Nov. 4, 2004

(54) Method of cell analysis using small volume of blood

(76) Inventor: Georges Puzant Der-Balian, Mountain View, CA (US)

Correspondence Address: Aldo D. Test, DORSEY & WHITNEY LLP, Suite 3400, 4 Embarcadero Center, San Francisco, CA, 94111, US

(21) Appl. No.: 10/426,386

(22) Filed: Apr. 29, 2003

(51) Int. Cl. G01N 33/53

(52) U.S. Cl. 435/7.2

(57) ABSTRACT

A method of performing white blood sub cell analysis is described which can be practiced using small volumes of blood. The sample is caused to flow through a capillary past a detection region where tagged or labeled cells fluoresce. The fluorescence is detected to provide a count of the labeled cells.

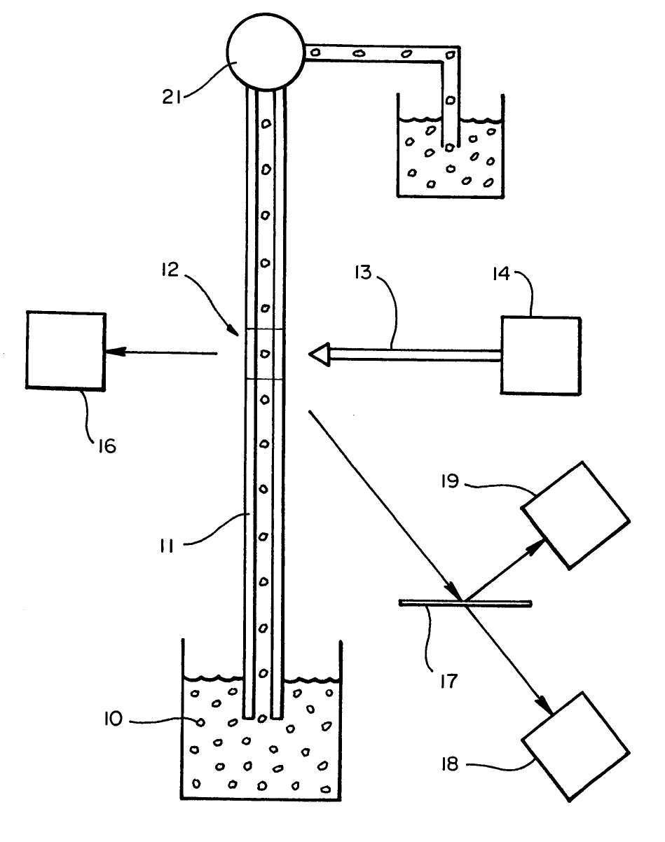

### BRIEF DESCRIPTION OF THE INVENTION

This present invention relates to a method of cell analysis using a very small number of cells and/or volumes of blood.

### BACKGROUND OF THE INVENTION

A common problem in cell biology is that there is often a minimal number of cells or volume of sample available with which to perform analyses. A particularly pertinent example is the study of different white cell subsets in blood. If large volumes of blood are available for analysis (e.g. 50 uL and above) traditional flow cytometry can be used with labeled antibodies to specific markers (such as CD3) to count the cells and estimate the relative numbers of different white cell subsets in a sample of blood. The problem is that in many situations the amount of blood available is very small. Examples include the very small volumes of blood typically available from human infants, animal infants, small animals and other species used in life science research such as mice, rats and zebrafish. Additionally, in many situations in the veterinary arena only small volumes of blood will be available from small animals, fish, reptiles and other organisms. In some examples the limited volume of sample may not be blood but may be, for example, cell preparations from small organisms such as C. elegans (a worm commonly used in research) or Drosophila larvae and other liquid body samples harvested in the clinical environment (e.g. amniotic fluid, cerebrospinal fluid). These situations require technology that can analyze minute quantities of material. The need is for a procedure and an instrument system that is flexible, robust and enables the generation of reliable results from small volumes in a cost effective and easy to use manner.

The capability to analyze small blood volumes is particularly relevant in situations where scientists have specifically bred model animals (e.g. transgenic, in-bred or gene knock-out organisms) which are very expensive or in very short supply. In these cases taking small volumes of blood from a living organism for analysis will allow characterization of the blood cell population without sacrificing the animal.

Furthermore, because it is only possible to take very small blood samples from model organisms such as mice, rats and zebrafish without sacrificing them these model organisms are not typically used for longitudinal studies where samples are taken at time intervals from a single animal. The innovation described in this disclosure will allow such longitudinal studies to be performed.

### OBJECTS AND SUMMARY OF THE INVENTION

It is an object of the present invention to provide a method of performing white blood cell sub setting employing small volumes of blood.

There is provided a method of performing white cell sub setting analysis on small volumes of blood comprising the steps of applying CD marker specific fluorescently labeled antibodies to the blood sample, then aspirating the sample through a capillary, illuminating a specific volume of the capillary, and detecting the fluorescence from labeled cells as they pass through the specific volume to identify the cell subsets.

### BRIEF DESCRIPTION OF THE DRAWINGS

The invention will be more clearly understood from the following description when it is read in conjunction with the accompanying drawing.

### DESCRIPTION OF THE PREFERRED EMBODIMENTS

The cell suspension 10 to be analyzed is aspirated through a capillary 11 as shown in the FIGURE. The cell suspension flows past analyzing volume 12 with the cells singulated. The cells which have been labeled with a CD marker antibody are excite by light beam 13 from a suitable source 14. Scattered light is detected by a detector 16 which is used to provide a count of all cells which have traversed the volume whether labeled or not. Cells which have been tagged or labeled with a CD specific antibody emit light at a corresponding wavelength. The light emitted by tagged cells is applied to a beam splitter 17. The beam splitter transmits light having wavelengths above a given wavelength to a detector assembly 18, which may include a filter, and reflects emitted light having wavelengths below the given wavelength to a detector assembly 19 which may include a filter. As a result cells with two different labels can be detected. The end of the capillary is immersed in the sample aliquot and sample is aspirated through the capillary by a pump 21.

A series of experiments were conducted to determine the volume of blood required to obtain repeatable sub cell counts using the above described apparatus as compared to the volume of blood required to obtain repeatable sub cell counts using a conventional flow cytometer such as the FACS Cytometer sold by Becton Dickinson.

The FACS experiment used 50 microliters of blood per assay. One isotype control tube (isos) and eight assay tubes were used. Each tube received 5 microliters of Cy-Chrome or Phycoerythrin (PE) labeled antibody. The isos tube got 5 microliters of Cy-Chrome and 5 microliters of PE labeled control isotypes. The tubes were incubated at room temperature for 10 minutes. 1 ml of PharMlyse was added to lyse the red cells. The cells were vortexed. After 10 minutes at room temperature the cells were washed by centrifugation with Buffer (PBS). The cells were resuspended in 250 microliters PBS and read on a FACS Calibur sold by Becton Dickinson. At least 6,000 cells were counted.

Seven other experiments were run as follows: for experiments 1, 2, 6, and 7, ten microliters of blood were added to PBS to a total volume of 200 microliters; 20 microliters were then distributed into each of 9 Eppendorfs; for experiments 4 and 5, two and one half microliters of blood were added to 97.5 microliters PBS. Then ten microliters of the blood PBS mixture were dispensed in 9 Eppendorfs. In all cases 1.5 microliter of labeled antibodies were added to the tube. For experiment 3, five microliters of blood was added to 95 microliters of PBS. The tubes were incubated for 10 minutes at room temperature. Two hundred microliters of PharMlyse were added and the tubes were vortexed and read on the above described instrument available from Guava Technologies, Inc. Model No. ______. For experiments 4 and 5, only 100 microliters PharMlyse were used. At least 2,000 cells were counted but in experiment 4 only 500 cells were counted.

Table 1 shows the percentage of cell subsets in the blood sample using the listed CD antigens for seven experiments using the microliters of blood shown at the bottom of each column.

TABLE 1

| CD | | | Exp | Exp | Exp | Exp | Exp | |
|---|---|---|---|---|---|---|---|---|
| antigens | FACS | Exp 1 | 2 | 3 | 4 | 5 | 6 | Exp 7 |
| Isos | 0.05 | 0.2; | 2.4; | 0.4; | 0.9; | 0.1; | 0.1; | 0.2; |
| | | 0.2 | 2.1 | 1.6 | 1.5 | 0.5 | 0.5 | 0.6 |
| CD3 | 21.6 | 21.6 | 21.2 | 20.5 | 21.6 | 22.5 | 21.6 | 21.8 |
| CD4 | 12.4 | 13.5 | 12.1 | 11.9 | 13.0 | 13.9 | 12.4 | 12.6 |
| CD8 | 9.4 | 8.1 | 8.2 | 8.8 | 9.0 | 9.3 | 9.2 | 8.4 |
| CD14 | 4.5 | 6.1 | 4.8 | 4.3 | 4.4 | 4.3 | 4.6 | 4.1 |
| CD15 | 54.9 | 49.7 | 50.5 | 54.3 | 50.0 | 51.2 | 55.5 | 57.9 |
| CD19 | 2.3 | 2.9 | 2.6 | 2.6 | 3.5 | 2.8 | 3.4 | 3.5 |
| CD45 | 99.3 | 98.5 | 96.8 | 96.8 | 94.4 | 96.2 | 94.7 | 97.1 |
| CD56 | 12.3 | 12.5 | 10.8 | 10.4 | 12.7 | 12.7 | 11.2 | 12.8 |

Microliters of blood used per experiment

| | | | | | | | | |
|---|---|---|---|---|---|---|---|---|
| Blood | 450 | 10 | 10 | 5 | 2.5 | 2.5 | 10 | 10 |

It is clear from this data that the volume of blood to carry out a study is between 0.6 and 2.5 percent of that needed for carrying out a study using conventional cytometry. This permits studies that have heretofore not possible.

It is apparent that the described method can be employed to perform immunophenotyping or identification of molecules that are associated with various types of cells, such as, lymphoma, leukemia and AIDS cell by using suitable immunophenotypic markers (antibodies) which fluoresce when exposed to light.

1. A method of analyzing cells labeled with a fluorescent label, wherein the cells are obtained from a small volume of sample comprising:
detecting fluorescence from the labeled cells as the cells are drawn through a microcapillary cytometer optically coupled to a fluorescence system.

2. The method of claim 1 wherein the labeled cells are subsets of white blood cells.

3. The method of claim 2 wherein the fluorescent label labels a cluster designation (CD) antigen.

4. The method of claims 1, 2 or 3 including the step of detecting light scattered by the cells to thereby count the total number of cells.

5. The method of claims 1, 2 or 3 in which the cells are drawn through the microcapillary by dipping one end of the microcapillary in a solution containing the cells and aspirating the solution through the microcapillary.

6. A method of performing white blood cell subset analysis using cells obtained from a small volume of blood comprising:
labeling the cells with subset specific antibodies, wherein each antibody subset comprises a distinguishable fluorescent label and
detecting the fluorescence signal emitted by the labeled cells as the cells are drawn through a microcapillary cytometer optically coupled to a fluorescent system, whereby the white blood cell subsets are analyzed.

7. The method of claim 6 including detecting light scattered by all cells to provide a count of all blood cells.

8. The method claims 6 or 7 wherein each antibody subset is to a cluster designation (CD) antigen.

9. The method of claims 6 or 7 wherein the volume of blood is a microvolume.

10. The method of claim 1 wherein the sample is blood.

11. The method of claim 1 wherein the sample is obtained from a small animal.

12. The method of claim 11 wherein said small animal is a mouse.
